(19) United States
(12) Patent Application Publication
(10) Pub. No.: US 2015/0165152 A1
(43) Pub. Date: Jun. 18, 2015

(54) ARTICULATING HEADGEAR ATTACHMENT

(71) Applicant: KONINKLIJKE PHILIPS N.V., EINDHOVEN (NL)

(72) Inventor: Richard Thomas Haibach, Verona, PA (US)

(21) Appl. No.: 14/418,125

(22) PCT Filed: Jul. 22, 2013
(86) PCT No.: PCT/IB2013/055991

§ 371 Date: Jan. 29, 2015

Related U.S. Application Data
(60) Provisional application No. 61/678,174, filed on Aug. 1, 2012.

(51) Int. Cl. A61M 16/06; A62B 18/08

(52) U.S. Cl. CPC A61M 16/0683; A62B 18/084; A61M 2205/0216; A61M 2209/088

(57) ABSTRACT

A latch headgear attachment mechanism for a respiratory mask. The headgear attachment comprising a support assembly (40) for a respiratory interface device (8), the support assembly having a coupling assembly (50) for a mask (10). The coupling assembly includes at least one elongated stand-off member (52) having a first end (54) and a second end (56). The stand-off member first end has a pivot assembly (60). The stand-off member second end has a strap coupling (58). The stand-off member first end pivot assembly is pivotally coupled to the mask. Thus, the stand-off member may move between a first configuration, wherein the stand-off member second end is spaced from the mask (and visible to the user), and a second configuration, wherein the stand-off member second end is disposed adjacent to the mask (and tucked out of the way).

### CROSS-REFERENCE TO RELATED APPLICATIONS

This patent application claims the priority benefit under 35 U.S.C. §119(e) of U.S. Provisional Application No. 61/678,174 filed on Aug. 1, 2012, the contents of which are herein incorporated by reference.

### BACKGROUND OF THE INVENTION

1. Field of the Invention

The present invention relates to respiratory interface devices for transporting a gas to and/or from an airway of a user which include, but not limited to, a mask having a support assembly with straps and, in particular, to a mask having pivotable support assembly couplings that may be pivoted outwardly so as to enable the user to see the coupling while attaching a strap thereto and pivoted inwardly to have a reduced profile.

2. Description of the Related Art

A variety of respiratory masks are known which cover the areas surrounding the nose and/or mouth of a human user and that are designed to create an effective fit against the user's face. Typically, gases can be provided at a positive pressure within the mask for consumption by the user. The uses for such masks include high altitude breathing (aviation applications), swimming, mining, fire fighting, and various medical diagnostic and therapeutic applications.

One requisite of many of these masks, particularly medical respiratory masks, is that they provide an effective fit against the user's face and that the mask contours with the user's face to limit or prevent leakage of the gas being supplied. The fit of a mask is partially controlled by the location of the couplings of the support assembly for the mask. That is, the support assembly for the mask is, typically, one or more straps, or other devices, that encircle the user's head. The support assembly couplings may attach at various locations about, or near, the perimeter of the mask. For example, for an oral/nasal mask, the couplings are disposed on either side of the mask body, i.e. over the user's cheeks. Additional couplings may be disposed on a forehead pad that is coupled to the mask by a vertical member. A nasal mask may include an elongated member that extends generally horizontally under the user's nose; a strap is coupled to each end of the elongated member.

The location for the support assembly couplings to provide the best fit, however, may not always correspond to a convenient location for the support assembly couplings. For example, a user may leave the support assembly coupled to the mask while putting the mask on. This is often inconvenient as the support assembly is not much larger than the user's head, meaning the support assembly is tight, and the straps may become twisted while pulling it into place. If the user decouples one side of the support assembly, the user may position the mask and wrap the support assembly around their head. Then, however, the user must couple the support assembly to a coupling that is out of, or just barely in, sight. That is, most users cannot see the coupling that is spaced less than about an inch out from their cheek.

The support assembly for a nasal mask suffers from the same disadvantage. That is, the coupling is not in an easily visible location. Moreover, the support for a nasal mask suffers an additional disadvantage, although it is too close to see, the support does extend far enough away from the user's face to catch on other objects. That is, a sleeping user who rolls their head to the side is likely to catch the support on a pillow and dislodge the nasal mask.

### SUMMARY OF THE INVENTION

One object of the invention is to provide a support assembly coupling assembly for a respiratory interface device mask including at least one elongated stand-off member having a first end and a second end. The stand-off member first end has a pivot assembly. The stand-off member second end has a strap coupling. The stand-off member first end pivot assembly is pivotally coupled to the mask. Thus, the stand-off member may move between a first configuration, wherein the stand-off member second end is spaced from the mask (and visible to the user), and a second configuration, wherein the stand-off member second end is disposed adjacent to the mask (and tucked out of the way).

Another object of the invention is to provide a support assembly coupling assembly for a respiratory interface device mask that includes a flexible body having a first end, a second end, and a mask coupling. The flexible body is structured to move between a first configuration, wherein the flexible body first and second ends are spaced from the mask, and a second configuration, wherein the flexible body first and second ends are disposed adjacent to the mask.

Another object of the invention is to provide a support assembly for a respiratory interface device mask that includes a first elongated, curved stand-off member and a second elongated, curved stand-off member. The first elongated, curved stand-off member has a first end, a second end, a pivot assembly and a coupling. The first stand-off pivot assembly is disposed at the first stand-off member first end. The first elongated, curved stand-off member has a sufficient length to extend to the back of a user's head. The second elongated, curved stand-off member has a first end, a second end, a pivot assembly and a coupling. The second stand-off member pivot assembly is disposed at the second stand-off member first end. The second elongated, curved stand-off member has a sufficient length to extend to the back of a user's head. Each stand-off member first end pivot assembly is pivotally coupled to the mask. Thus, the first and second stand-off members may move between a first configuration, wherein the first and second stand-off member second ends are disposed in front of the mask, and a second configuration, wherein the first and second stand-off members form a loop with the mask.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1 is an isometric view of a respiratory interface device with a first embodiment of a mask and support assembly coupling assembly;

FIG. 2 is a bottom view of the respiratory interface device of FIG. 1;

FIG. 3. is a bottom view of a respiratory interface device with another embodiment of a mask and support assembly coupling assembly in a first configuration;

FIG. 4. is a top view of a respiratory interface device of FIG. 3 in a second configuration;

FIG. 5 is a detail schematic view of a support assembly coupling assembly;

FIG. 6. is a front view of a respiratory interface device with another embodiment of a mask and support assembly coupling assembly;

FIG. 7 is an isometric view of the respiratory interface device of FIG. 6;

FIG. 8 is a detail schematic view of another support assembly coupling assembly;

FIG. 9 is a top view of a respiratory interface device with another embodiment of a mask and support assembly coupling assembly in a first configuration; and

FIG. 10. is a top view of a respiratory interface device of FIG. 9 in a second configuration.

### DETAILED DESCRIPTION OF EXEMPLARY EMBODIMENTS

As used herein, the singular form of “a,” “an,” and “the” include plural references unless the context clearly dictates otherwise. As used herein, the statement that two or more parts or components are “coupled shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

As used herein, “a generally continuous seal” may have a gap or may gap when the user moves. As used herein, “a more complete seal” has a gap that is shorter in length than a gap of a generally continuous seal, or, is resistant to gapping when the user moves.

As used herein, “correspond” indicates that two structural components are sized to engage each other with a minimum amount of friction. Thus, an opening which corresponds to a member is sized slightly larger than the member so that the member may pass through the opening with a minimum amount of friction. This definition is modified if the two components are said to fit “snugly” together. In that situation, the difference between the size of the components is even smaller whereby the amount of friction increases. If the element defining the opening and/or the component inserted into the opening are made from a deformable or compressible material, the opening may even be slightly smaller than the component being inserted into the opening.

As used herein, a “coupling” or a “coupling component” is one element of a coupling assembly. That is, a coupling assembly includes at least two elements, or components, that are structured to be coupled together. It is understood that the elements of a coupling assembly correspond to each other or are otherwise structured to be joined together. For example, in a coupling assembly, if one coupling element is a bolt, the other coupling element is a nut. Further, it is understood that the two elements of a coupling assembly may not be described at the same time.

As used herein, “field of vision” means the entire expanse of space visible to the user's eyes, including areas that are visible in a peripheral area. It is understood that a user may move their eyes to adjust their field of vision. It is further understood that clarity is not required for an object to be in a user's “field of vision.” That is, an object so close to a user's eye as to be seen as blurry is still within the user's “field of vision.”

As used herein, “substantially outside of the user's field of vision” means areas that are not in, or are only partially in, the user's “field of vision.” Thus, an object that is partially visible within a peripheral area but not entirely visible is “substantially outside of the user's field of vision.” Further, specific examples of elements that are “substantially outside of the user's field of vision” are provided below and it is understood that the term “substantially outside of the user's field of vision” specifically applies to such elements.

As used herein, “mounting” or “mounted” means a coupling that is permanent. That is, the “mounted” element is not intended to be removed from that which it is mounted on. A temporary coupling, such as, but not limited to a clip or snap, is not a mounting. As used herein, “pivot” means a coupling that allows an element coupled to the pivot to move through an arc, similar to a light switch.

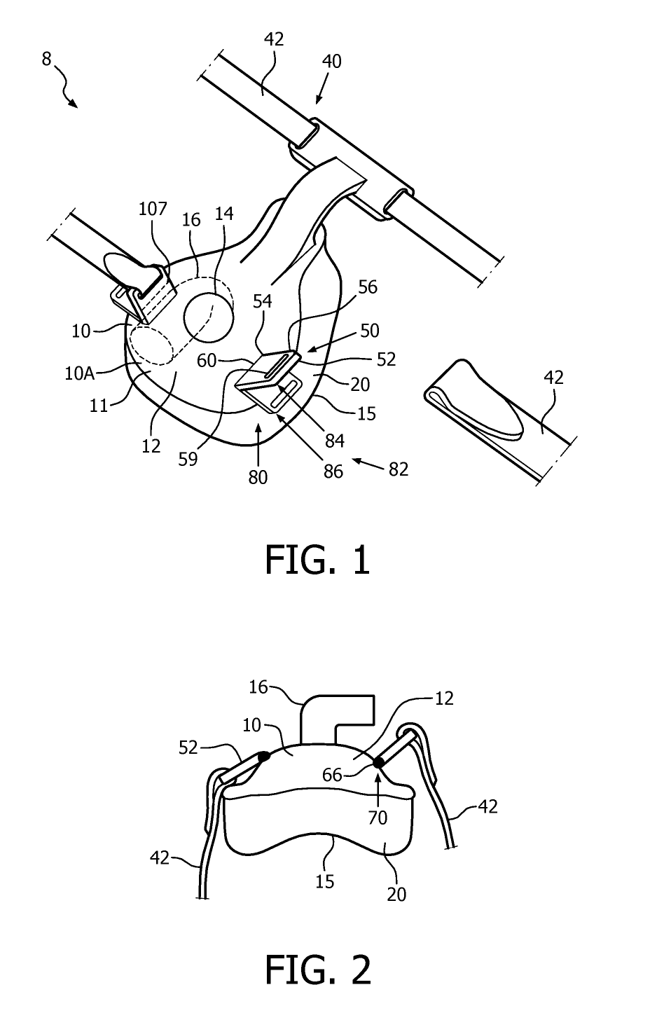

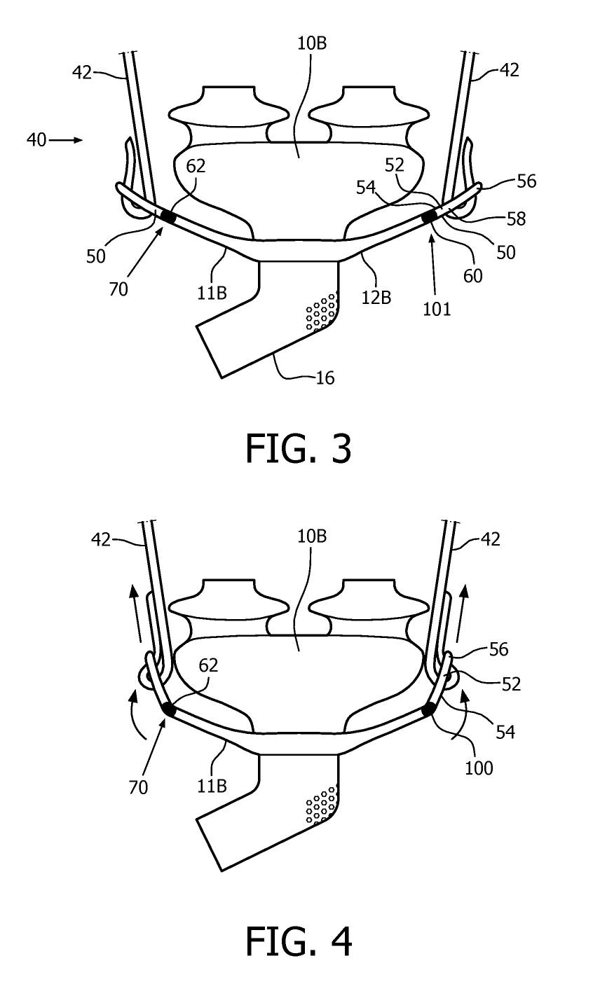

FIGS. 1 and 2 show a respiratory interface device 8 according to an exemplary embodiment of the invention. Respiratory interface device 8 includes a respiratory mask 10 and a support assembly 40. FIG. 1 shows an oral/nasal mask 10A, structured to extend over a user's mouth and nose. FIGS. 3 and 4 show a nasal mask 10B structured to interface with a user's nose. Such a mask is also referred to as a nasal cannula or nasal prongs type of patient interface. The components of oral/nasal mask 10A and nasal mask 10B have similar purposes and like reference numbers will be used below where possible. Where the components of oral/nasal mask 10A and nasal mask 10B are different, a reference number followed by the letter “A” will be used to indicate that the component is for oral/nasal mask 10A, and, a reference number followed by the letter “B” will be used to indicate that the component is for nasal mask 10B.

Mask 10 is coupled to a pressure generating system (not shown) via a patient circuit, as is conventionally known in the art. For purposes of the present invention, the pressure generating system is any device capable of generating a flow of breathing gas or providing gas at an elevated pressure. Examples of such pressure generating systems include a ventilator, CPAP device, or variable pressure device, e.g. an auto-titrating device, proportional assist ventilation (PANTO) device, proportional positive airway pressure (PPAP) device, C-Flex™ device, Bi-Flex® device, or a BiPAP® device manufactured and distributed by Philips Respironics of Murrysville, Pa., in which the pressure provided to the patient varies with the patient's respiratory cycle so that a higher pressure is delivered during inspiration than during expiration, or other pressure support device.

Respiratory mask 10 includes a body 11 with a faceplate 12 and a cushion 15, discussed below. In an exemplary embodiment, faceplate 12 is substantially rigid. In one exemplary embodiment, shown in FIG. 1, faceplate 12A is a single piece structured to cover the user's nose and mouth. That is, mask 10A has a peripheral contour that is structured to extend over a user's nose and mouth. In this embodiment, body 11 is coextensive with faceplate 12A. In another exemplary embodiment, mask 10B is structured to extend over just the user's nose. In this embodiment, an elongated body 11A is structured to extend generally horizontally under the user's nose.

Faceplate 12 defines lower opening 14. Lower opening 14 can function as a gas inlet. Gas inlet (lower opening 14) can be coupled to a coupling device 16, such as a swivel conduit, for carrying gas such as air between mask 10 and an external gas source (not shown), such as a blower, or any other suitable device. It is contemplated that the external gas source can encompass, without limitation, any gas delivery or gas generation system capable of supplying gas for consumption by a user. Non-limiting examples of various gas delivery therapies can include but are not limited to continuous positive airway pressure (CPAP) therapy, auto-titration positive airway pressure therapy, and bi-level positive airway pressure (BiPAP) therapy, as noted above. The particular coupling device 16, shown in ghost in FIG. 1, is not meant to be limiting and it should be understood that the present invention contemplates a variety of different coupling devices that could be attached, either permanently or selectively, to lower opening 14 to carry gas to or from mask 10. Thus, a variety of coupling devices (e.g., with or without swivels on one or both ends, and with or without an exhalation system formed integral to the device) may be substituted for coupling device 16.

Cushion 15 is structured to extend from faceplate 12 toward the user's face and generally defines the depth of mask 10. Cushion 15 includes a cushion body 20 made from a flexible material. Cushion 15 is structured to engage the user's face and provides a generally continuous seal. This seal may be improved to be a more complete seal if mask 10 is maintained in an orientation that is generally tangent relative to the user's face. The bias that causes cushion 15 to engage the user's face is created by support assembly 40.

In the exemplary embodiments shown in FIGS. 1-4, support assembly 40 includes at least one strap 42 structured to extend about the user's head. At least one strap 42 is selectively coupled to mask 10 by a strap coupling 58, described below. As is known, support assembly straps 42 may form a web (not shown) having straps 42 in any number of configurations. In an exemplary embodiment, and as shown in the FIG. 1, two straps 42 are used; the claims, however, are not limited to this configuration. Straps 42 may be made from an elastic or non-elastic material.

A support assembly coupling assembly 50, i.e. a coupling assembly on mask that is structured to be coupled to the support assembly 40, is disposed on each lateral side of mask 10. Each support assembly coupling assembly 50 is substantially similar and therefore only one of each support assembly coupling 50 will be discussed below. It is understood, however, that the description is applicable to each support assembly coupling assembly 50. Support assembly coupling assembly 50 includes at least one elongated stand-off member 52 having a first end 54 and a second end 56. Stand-off member first end 54 has a pivot assembly 60. Stand-off member second end 56 has a strap coupling 58. That is, strap coupling 58 is disposed on stand-off member 52 and does not comprise the entire stand-off member 52. As shown as an exemplary embodiment, strap coupling 58 is a slot 106, discussed below, in stand-off member second end 56 through which strap 42 may be looped. It is understood, however, that any type of coupling assembly may be used to couple strap 42 to mask 10. Such coupling assemblies may be, but not limited to, a snap assembly, a buckle, hook-and-bar, and a clip. It is understood that on coupling assembly component is coupled to mask 10 and the other is disposed on strap 42. As discussed in detail below, stand-off member first end pivot assembly 60 is pivotally coupled to, and more specifically mounted on, mask 10.

With this arrangement of elements, stand-off member 52 may move between a first configuration, wherein stand-off member second end 56 is spaced from mask 10, and a second configuration, wherein stand-off member second end 56 is disposed adjacent to mask 10. As used herein with respect to the first and second configurations, “spaced” and “adjacent” are relative terms. That is, when stand-off member 52 is in the first configuration, stand-off member second end 56 is further away from mask 10 than when stand-off member 52 is in the second configuration. Further, in an exemplary embodiment, when stand-off member 52 is in the first configuration, i.e. stand-off member second end 56 is spaced from mask 10, stand-off member second end 56 is within the user′ field of vision. In an exemplary embodiment, stand-off member second end 56 is spaced between about ⅜ inch and 1.5 inches from mask 10 when in the first configuration.

When stand-off member 52 is in the second configuration, i.e. stand-off member second end 56 is adjacent to mask 10, stand-off member second end 56 is substantially outside of the user′ field of vision. It is understood that objects that are close to a user's face, such as mask 10, and more specifically faceplate 12, are so close as to be only partially visible to the user. Thus, as used herein, a coupling disposed on mask 10, and more specifically faceplate 12, that may not move away from mask 10 will be “substantially outside of the user's field of vision.” It is specifically noted that a coupling, such as, but not limited to, a loop, may be pivotally and directly coupled to mask 10. The side of such a loop that is not coupled to mask 10 may pivot away from mask 10; the entire coupling, however, may not. Such a coupling is not a “stand-off member” as used herein. That is, as used herein, a “stand-off member” is a pivotable element that is mounted on mask 10 and is structured to provide spacing for another element such as a coupling.

It is understood that one component of the pivot assembly does not pivot with the “stand-off member.” Further, a coupling may not arbitrarily be divided so as to be said to have a stand-off portion and a coupling portion. For example, a loop, that is the entire loop, is a “coupling” and does not include a “stand-off member” as no part of the loop is structured to provide spacing independent of the structure of the loop. Thus, as used herein a portion of a coupling is not a “stand-off member.” Further, as used herein, a coupling that is directly coupled to mask 10, i.e. without a stand-off member between the coupling and mask 10, is “substantially outside of the user's field of vision.”

Thus, when stand-off member 52 is in the first configuration, a user may see stand-off member second end 56, and moreover the user may see strap coupling 58. That is, stand-off member second end 56 is no longer disposed adjacent to, or on, mask 10. This allows the user to more easily couple strap 42 to strap coupling 58. After the strap 42 is coupled to strap coupling 58, the user moves stand-off member 52 to the second configuration, wherein stand-off member second end 56, strap coupling 58 and strap 42, are substantially outside of the user's field of vision. Further, in the second configuration, stand-off member 52 has a reduced profile. That is, stand-off member 52 does not extend a substantial distance from mask 10 and is less prone to contact other objects.

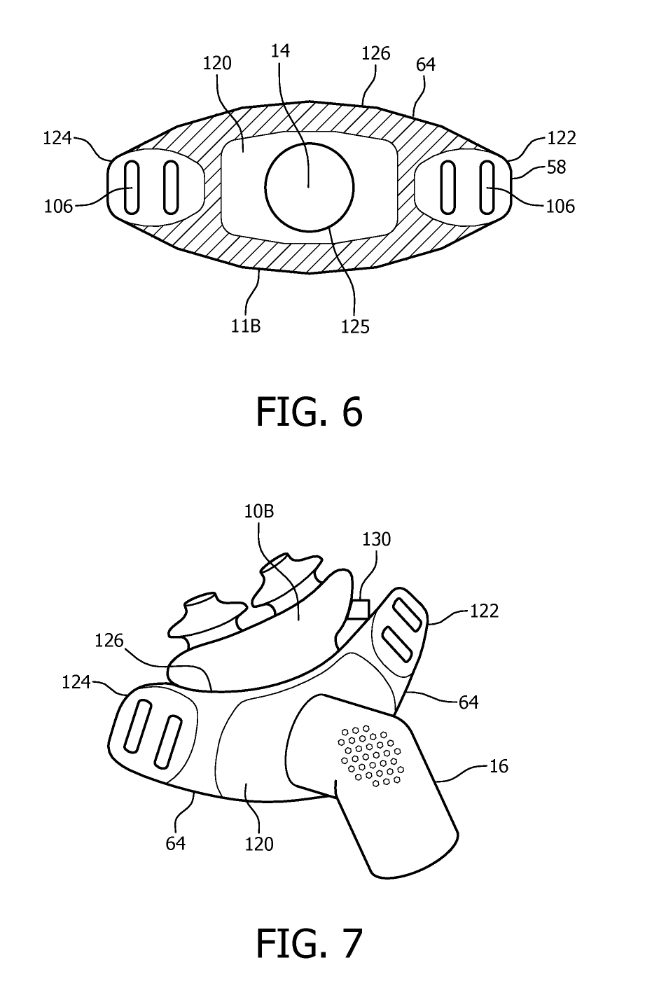

In exemplary embodiments, pivot assembly 60 includes one of a molded hinge 62 (FIG. 3), a flexure 64 (FIG. 6), and barrel hinge 66 (FIG. 2). It is noted, however, that any other type of hinge or pivot assembly may be used. As used herein, a “molded hinge” is a localized flexible portion of a molded material and includes a living hinge. As used herein, a “flexure” is a non-localized flexible portion of a material; that is, a “flexure” is that portion of a flexible material that bends when a bias is applied thereto. As used herein, a “barrel hinge” includes any hinge having a loop coupled to a moving element and a pin extending through the loop; the loop may be an opening in the moving element. Pivot assembly 60 is structured to allow stand-off member 52 to rotate about at least one axis and relative to mask 10.

Pivot assembly 60 may include a biasing device 70. Biasing device 70 is structured to bias the stand-off member 52 to one of the first or second configurations. That is, in one exemplary embodiment (FIG. 3), biasing device 70 is structured to bias stand-off member 52 to the first configuration. This positions stand-off member second end 56 within the user's field of vision unless another bias is applied to stand-off member 52. Another bias may be provided by strap 42. That is, stand-off member 52 is biased to the first configuration so that a user may attach strap 42 to strap coupling 58. The user then tightens strap 42 (or strap 42 may automatically tighten if elastic) thereby overcoming the bias of biasing device 70 and moving stand-off member 52 to the second configuration.

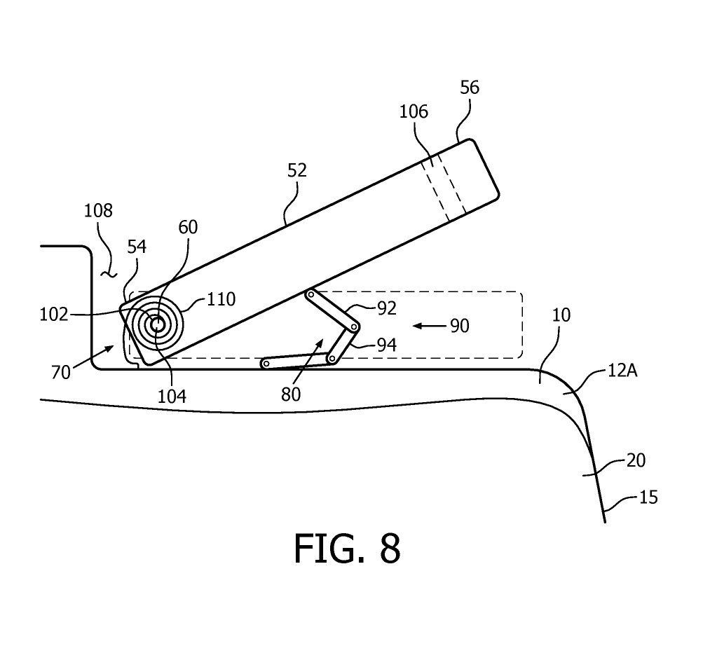

In an alternate embodiment (FIG. 8), biasing device 70 is structured to bias stand-off member 52 to the second configuration. In this embodiment, biasing device 70 helps ensure that stand-off member 52 remains out, or substantially out, of the user's field of vision. The type of biasing device 70 used is related to the type of pivot assembly 60. For example, molded hinge 62 or flexure 64 may utilize biasing device 70 formed by pre-stressing of the molded material forming molded hinge 62 or flexure 64. Alternatively, barrel hinge 66 may utilize biasing device 70 such as, but not limited to, a torsion spring 110 (discussed below) disposed about the hinge pin 104 (discussed below), or, a compression spring (not shown) disposed between mask 10 and stand-off member 52.

Pivot assembly 60 may include a locking assembly 80. Locking assembly 80 is structured to temporarily maintain stand-off member 52 in one of the first configuration or the second configuration. For example, if biasing device 70 is structured to bias stand-off member 52 to the first configuration, locking assembly 80 may temporarily lock stand-off member 52 in the second configuration thereby reducing stress on an elastic strap 42 and help ensure that stand-off member 52 remains out, or substantially out, of the user's field of vision. Thus, locking assembly 80 may include a ball-and-detent assembly 82 where, in an exemplary embodiment, a hemisphere or ball 84 is disposed on stand-off member 52 and a detent 86 is disposed on mask 10.

When stand-off member 52 is in the second configuration, ball 84 is disposed in detent 86. In another exemplary embodiment, locking assembly 80 may temporarily lock stand-off member 52 in the first configuration thereby allowing the user to couple strap 42 to strap coupling 58. That is, if biasing device 70 is structured to bias stand-off member 52 to the second configuration, locking assembly 80 may include a toggle assembly 90 having a first toggle link 92 and second toggle link 94. Toggle assembly 90 is moved into an over-toggle, i.e. a temporarily locked configuration, when stand-off member 52 is disposed in the first configuration. This allows a user to more easily couple strap 42 to strap coupling 58 in that the user does not have to hold stand-off member 52 in the first configuration. After strap 42 is coupled to strap coupling 58, the user actuates the toggle assembly 90, i.e. moves the first and second links 92, 94 out of the over-toggle position thereby allowing toggle assembly 90 to collapse and allowing biasing device 70 to bias stand-off member 52 to the second configuration.

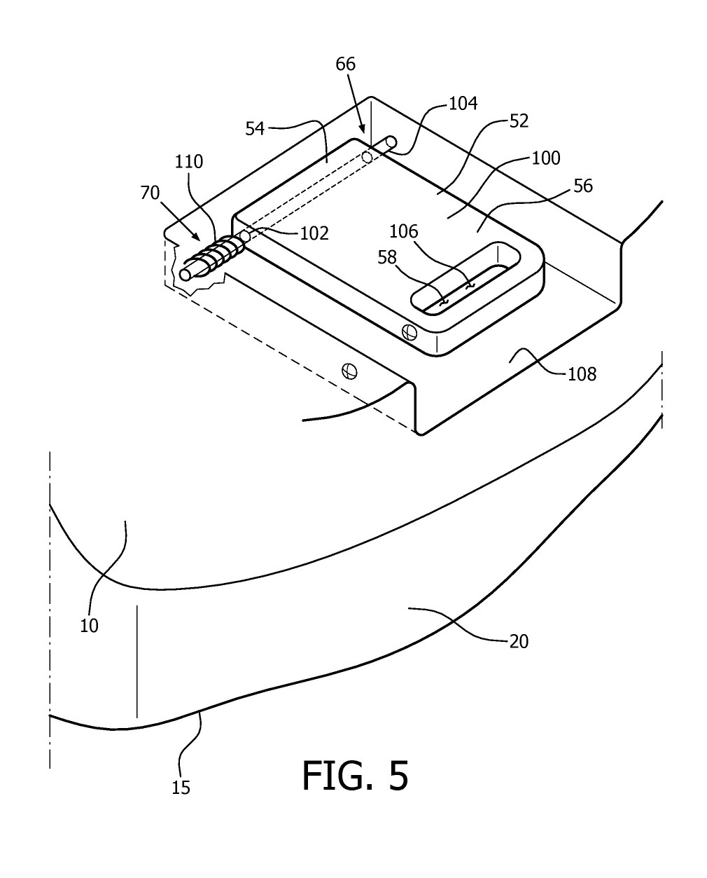

In an exemplary embodiment, shown schematically in FIG. 5, an oral/nasal mask 10A includes pivot assembly 60 with biasing device 70 structured to bias stand-off member 52 to the first position and locking assembly 80 structured to temporarily lock stand-off member 52 in the second position. That is, stand-off member 52 has a planar body 100. As shown, planar body 100 includes a barrel hinge 66 having an opening 102 in planar body 100 and a pin 104 extending from mask 10. Opening 102 extends in the plane of planar body 100. Pin 104 is disposed through opening 102, thereby pivotally coupling planar body 100 to mask 10. As noted above, stand-off member second end 56 has a strap coupling 58. In this embodiment, strap coupling 58 is material defining a slot 106 through which a strap 42 may be looped. It is noted that strap coupling 58, e.g. the material defining slot 106, can be distinguished from body 100 as shown at reference line 107 (FIG. 1). As noted above, stand-off member second end 56 is spaced between about ⅜ inch and 1.5 inches from mask 10 when in the first configuration. Thus, in an exemplary embodiment, the combined length of body 100 and strap coupling 58 is between about ⅜ inch and 1.5 inches.

Mask 10 may include a pocket or recess 108 in which planar body 100 may be disposed, or partially disposed, when stand-off member 52 is in the second configuration. Further, biasing device 70 includes a torsion spring 110 disposed about pin 104 and coupled to both planar body 100 and mask 10. Torsion spring 110 is structured to bias planar body 100 away from mask 10. That is, torsion spring 110 is structured to bias stand-off member 52 to the first configuration. Further, locking assembly 80 includes ball-and-detent assembly 82 with ball 84 disposed on an edge of planar body 100 and detent 86 in recess 108. When stand-off member 52 is in the second configuration, ball 84 is disposed in detent 86.

In another exemplary embodiment, shown in FIGS. 3 and 4, a nasal mask 10B includes an elongated body 11B structured to extend generally horizontally under the user's nose, as noted above. At least one lateral end, and in an exemplary embodiment both lateral ends, of elongated body 11B include support assembly coupling assembly 50. In this embodiment, pivot assembly 60 is molded hinge 62. Pivot assembly 60 includes biasing device 70 structured to bias stand-off member 52 to the second configuration. Biasing device 70 is in the form of pre-stressed molded material forming molded hinge 62. That is, during the formation of molded hinge 62, the molded material is pre-stressed to position stand-off member 52 in the second configuration. After the molding process is complete, molded hinge 62 will bias stand-off member 52 to the second configuration. It is noted that when using molded hinge 62, the material comprising molded hinge 62 is part of stand-off member 52. That is, molded hinge 62 may be disposed adjacent strap coupling 58 as the material of molded hinge 62 is part of stand-off member body 100. Thus, stand-off member 52, which includes molded hinge 62, is structured to provide spacing for another element such as a coupling.

In another exemplary embodiment, shown in FIGS. 6 and 7, another nasal mask 10B (FIG. 7) is shown. In this embodiment, elongated body 11B includes rigid portions 120, 122, 124 but is otherwise a substantially flexible body 126. Rigid portions 120, 122, 124 include a central portion 120, a first lateral portion 122 and a second lateral portion 124, each lateral portion 122, 124 disposed at one lateral end of body 11B. Central portion 120 defines lower opening 14 which acts as a mask coupling 125. First and second lateral portion 122, 124 define slots 106 that act as strap couplings 58. The remainder of body 11B is made from a flexible material. Thus, flexible body 126 is flexure 64. That is, one lateral portion, e.g. first lateral portion 122 and flexible body 126, collectively form one stand-off member 52 with flexible body 126, i.e. flexure 64, being pivot assembly 60. Thus, as shown, there are two stand-off members 52 wherein flexible body 126, i.e. flexure 64, acts as pivot assembly 60 for both stand-off members 52. As with molded hinge 62, discussed above, flexible body 126, i.e. flexure 64, may be pre-stressed so as to bias stand-off members 52 to one of the first or second configurations. Thus, pivot assembly 60 includes biasing device 70. Further, in this embodiment, the motion of stand-off member 52 is limited by a stop 130. Stop 130 extends from mask 10 and is structured to block the path of travel of stand-off member 52. A stop 130, shown schematically, may be used with any embodiment described above. For example, the bottom of recess 108 is also stop 130.

In another exemplary embodiment, shown schematically in FIG. 8, an oral/nasal mask 10A includes pivot assembly 60 with biasing device 70 structured to bias stand-off member 52 to the second position and locking assembly 80 structured to temporarily lock stand-off member 52 in the first position. That is, as before, stand-off member 52 has planar body 100. As shown, planar body 100 includes barrel hinge 66 having opening 102 in planar body 100 and pin 104 extending from mask 10. Opening 102 extends in the plane of planar body 100. Pin 104 is disposed through opening 102, thereby pivotally coupling planar body 100 to mask 10. As noted above, stand-off member second end 56 has strap coupling 58. In this embodiment, strap coupling 58 is slot 106 through which strap 42 may be looped.

Mask 10 may include pocket or recess 108 in which planar body 100 may be disposed, or partially disposed, when stand-off member 52 is in the second configuration. In this embodiment, however, biasing device 70, such as a torsion spring 110, is structured to bias stand-off member 52 to the second position. As such, locking assembly 80 is structured to temporarily maintain stand-off member 52 in the first configuration. In this embodiment, locking assembly 80 includes a toggle assembly 90 as described above. As shown in solid line, toggle assembly 90 is in an over-toggle position, thereby temporarily locking stand-off member 52 in the first configuration. As shown in ghost, toggle assembly 90 may be collapsed, thereby allowing locking stand-off member 52 to move to the second configuration under the bias of biasing device 70. It is noted that recess 108 may include a space for links 92 and 94, thereby allowing stand-off member 52, i.e. planar body 100, to rest flat in recess 108.

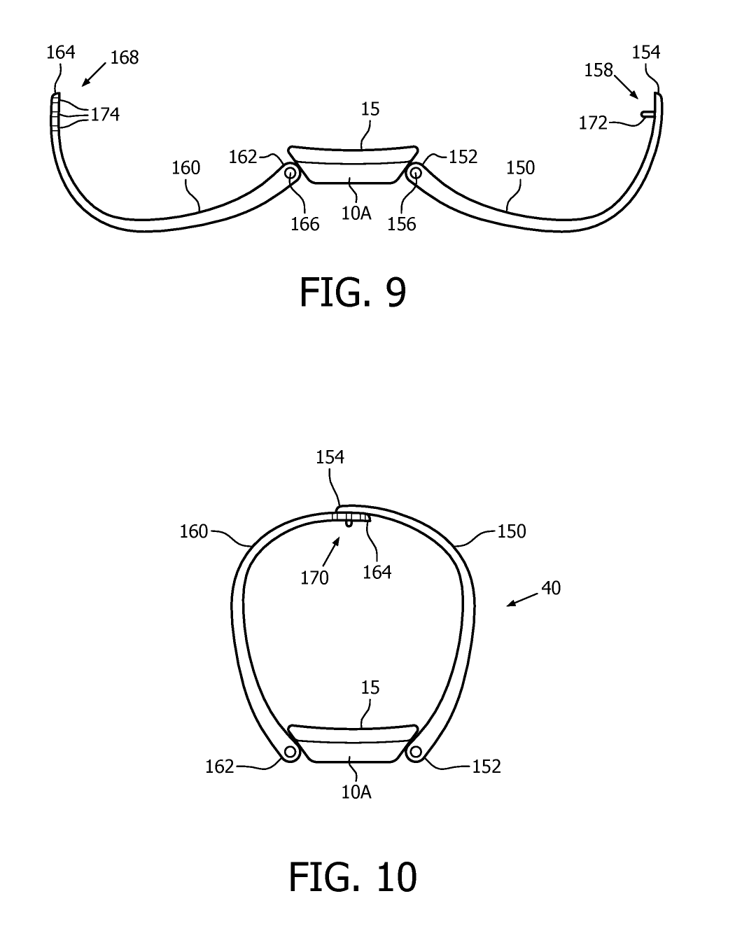

In another exemplary embodiment, shown in FIGS. 9 and 10, support assembly 40 may not include straps 42. That is, the function of straps 42 may be accomplished by providing support assembly 40 with a first and second elongated, curved stand-off member 150, 160. Each of first and second stand-off member 150, 160 have a first end 152, 162, a second end 154, 164, a pivot assembly 156, 166 and a coupling 158, 168. First and second curved stand-off members 150, 160 have a sufficient length and curvature to extend about a user's head with first and second stand-off member second ends 154, 164 meeting adjacent the back of the user's head. That is, first and second curved stand-off members 150, 160, together with mask 10, form a loop about the user's head.

First and second curved stand-off members pivot assemblies 156, 166 are similar to any of the pivot assemblies 60, 156, 166 described above. Each of the first and second curved stand-off members pivot assemblies 156, 166 are disposed at first and second stand-off member first ends 152, 162. As before, first and second curved stand-off members 150, 160 are structured to move between a first configuration and a second configuration. As shown in FIG. 9, in a first configuration, first and second stand-off members 150, 160 are substantially disposed in front of mask 10. In the second configuration, as shown in FIG. 10, first and second stand-off members 150, 160 form a loop with mask 10. That is, in use, first and second stand-off members 150, 160 encircle the user's head. Further, first and second curved stand-off members pivot assemblies 156, 166 may include a biasing device 70 structured to bias first and second stand-off members 150, 160 to one of the first or second configurations.

In this embodiment, rather than a strap coupling at each first and second stand-off member second end 154, 164, a coupling assembly 170 is provided. That is, coupling assembly 170 includes two components 172, 174 that are structured to be coupled together. In an exemplary embodiment, first and second stand-off member couplings 158, 168 are one of a hook-and-loop coupling and a peg-and-hole coupling. As used herein, a “peg-and-hole” includes at least one peg extending generally perpendicular to a first or second stand-off member second end 154, 164, and, at least one snugly corresponding opening in the other first or second stand-off member second end 154, 164. A “peg-and-hole” further includes a similar arrangement with a plurality of peg and openings, similar to the coupling often found on baseball caps. It is further noted that in this embodiment, stand-off member second ends 154, 164 may not be visible to the user when in the first configuration. This is acceptable as, in this embodiment, the user does not need to couple a strap 42 to stand-off member second ends 154, 164.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. (canceled)

2. A support assembly for a respirator interface device mask, the support assembly comprising:
at least one strap;
a support assembly coupling assembly having at ea one elongated stand-off member a ng a first end and a second end;
the stand-off member first end having a pivot assembly;
the stand-off member second end having a strap coupling;
the stand-off member first end pivot assembly is pivotally coupled to the mask; and
the at least one strap selectively coupled to the strap coupling, wherein the stand-off member may move between a first configuration, wherein the stand-off member second end is spaced from the mask, and a second configuration, wherein the stand-off member second end is disposed adjacent to the mask; and wherein the stand-off member first end pivot assembly includes a biasing device, and wherein the biasing device is structured to bias the stand-off member to one of the first or second configurations.

3. A support assembly for a respiratory interface device mask, the support assembly comprising;
at least one strap;
a support assembly coupling assembly having at least one elongated stand-off member having a first end and a second end;
the stand-off member first end having a pivot assembly;
the stand-off member second end having a strap coupling;
the stand-off member first end pivot assembly is pivotally coupled to the mask; and
the at least one strap selectively coupled to the strap coupling wherein the stand-off member may move between a first configuration, wherein the stand-off member second end is spaced from the mask, and a second configuration, wherein the stand-off member second end is disposed adjacent to the mask, wherein the stand-off member first end pivot assembly includes a biasing device and a locking assembly, wherein the locking assembly is structured to temporarily maintain the stand-off member in the first configuration; and wherein the biasing device structured to bias the stand-off member to the second configuration.

4. A support assembly for a respiratory interface device mask, the support assembly comprising:
at least one strap;
a support assembly coupling assembly having at least one elongated stand-off member having a first end and a second end;
the stand-off member first end having a pivot assembly;
the stand-off member second end having a strap coupling;
the stand-off member first end pivot assembly is pivotally coupled to the mask; and
the at least one strap selectively coupled to the strap coupling, Wherein the stand-off member may move between a first configuration, wherein the stand-off member second end is spaced from the mask, and a second configuration, wherein the stand-off member second end is disposed adjacent to the mask, and wherein the biasing device locking assembly is a toggle assembly.

5. A support assembly for a respiratory interface device mask, the support assembly comprising:
at least one strap;
a support assembly coupling assembly having at least one elongated stand-off member having a first end and a second end;
the stand-off member first end having a pivot assembly;
the stand-off member second end having a strap coupling;
the stand-off member first end pivot assembly is pivotally coupled to the mask; and
the at least one strap selectively coupled to the strap coupling, wherein the stand-off member may move between a first configuration, wherein the stand-off member second end is spaced from the mask, and a second configuration, wherein the stand-off member second end is disposed adjacent to the mask, and wherein the stand-off member first end pivot assembly includes a biasing device, and wherein the biasing device structured to bias the stand-off member to the first configuration.

6. The support assembly of claim 5, wherein the stand-off member first end pivot assembly includes a locking assembly structured to temporarily maintain the stand-off member in the second configuration.

7. The support assembly of claim 5, wherein the locking assembly is a ball-and-detent.

8. The support assembly of claim 5, wherein the pivot assembly includes a molded hinge; and wherein the biasing device is a pre-stressing of the molded hinge.

9. The support assembly of claim 5, further including a stop, the stop structured to resist movement past the second configuration.

10. A support assembly for a respiratory interface device mask, the support assembly comprising:
at least one strap;
a support assembly coupling assembly having at least one elongated stand-off member having a first end and a second end;
the stand-off member first end having a pivot assembly;
the stand-off member second end having a strap coupling;
the stand-off member first end pivot assembly is pivotally coupled to the mask;
the at least one strap selectively coupled to the strap coupling, wherein the stand-off member may move between a first configuration, wherein the stand-off member second end is spaced from the mask, and a second configuration, wherein the stand-off member second end is disposed adjacent to the mask; and wherein the pivot assembly is one of a molded hinge, a flexure, and barrel hinge.

11. The support assembly of claim 10, wherein the stand-off member first end pivot assembly includes a locking assembly structured to temporarily maintain the stand-off member in the second configuration.

12. The support assembly of claim 11, wherein the locking assembly is a ball-and-detent.

13. The support assembly of claim 10, further including a stop, the stop structured to resist movement past the second configuration.

14. A support assembly for a respiratory interface device mask, the support assembly comprising:
at least one strap;
a support assembly coupling assembly having at least one elongated stand-off member having a first end and a second end;
the stand-off member first end having a pivot assembly;
the stand-off member second end having a strap coupling;
the stand-off member first end pivot assembly is pivotally coupled to the mask;
the at least one strap selectively coupled to the strap coupling, wherein the stand-off member may move between a first configuration, wherein the stand-off member second end is spaced from the mask, and a second configuration, wherein the stand-off member second end is disposed adjacent to the mask, wherein the stand-off member includes at least one medial hinge disposed between the stand-off member first end and the stand-off member second end, and wherein at least one of the stand-off member first end pivot assembly and the stand-off member at least one medial hinge has a limited range of motion.

15. A support assembly for a respiratory interface device mask, the respiratory interface device including the mask, the support assembly comprising:
at least one strap;
a support assembly coupling assembly having a flexible body with a first lateral portion, a second lateral portion, and a mask coupling;
the at least one strap selectively coupled to the mask coupling; and
the flexible body structured to move between a first configuration, wherein the flexible body first and second lateral portions are spaced from the mask, and a second configuration, wherein the flexible body first and second ends are disposed adjacent to the mask.

16. The support assembly of claim 15, wherein the flexible body includes a biasing device, and wherein the biasing device is structured to bias the stand-off member to one of the first or second configurations.

17. The support assembly of claim 16, wherein the flexible body is made from a molded material, and wherein the biasing device is a pre-stressing of the flexible body.

18. A support assembly for a respiratory interface device mask, the support assembly comprising:
a first elongated, curved stand-off member having a first end, a second end, a pivot assembly and a coupling, the first stand-off member pivot assembly being disposed at the first stand-off member first end, the first elongated, curved stand-off member having a sufficient length to extend to the back of a user's head;
a second elongated, curved stand-off member having a first end, a second end, a pivot assembly and a coupling, the second stand-off member pivot assembly being disposed at the second stand-off member first end, the second elongated, curved stand-off member having a sufficient length to extend to the back of a user's head;
each stand-off member first end pivot assembly being pivotally coupled to the mask; and
wherein the first and second stand-off members may move between a first configuration, wherein the first and second stand-off members are substantially disposed in front of the mask, and a second configuration, wherein the first and second stand-off members form a loop with the mask.

19. The support assembly of claim 18, wherein the first and second stand-off member couplings are one of a hook-and-loop coupling and a peg-and-hole coupling.

20. The support assembly of claim 19, wherein:
the first stand-off member first end pivot assembly includes a biasing device;
the second stand-off member first end pivot assembly includes a biasing device; and
each biasing device structured to bias the stand-off members to one of the first or second configurations.

21. The support assembly of claim 19, wherein the first stand-off member first end pivot assembly is one of a molded hinge, a flexure, and barrel hinge, and wherein the second stand-off member first end pivot assembly is one of a molded hinge, a flexure, and barrel hinge.
